(19) United States
(12) Patent Application Publication
(10) Pub. No.: US 2004/0253317 A1
(43) Pub. Date: Dec. 16, 2004

(54) Pharmaceutical preparation for the treatment and diagnosis of tumors and method for the preparation of the lipid free fraction of blood plasma

(76) Inventor: Andras Bertha, Budapest (HU)

Correspondence Address: ANTHONY H. HANDAL, KIRKPATRICK & LOCKHART, LLP, 599 LEXINGTON AVENUE, 31ST FLOOR, NEW YORK, NY, 10022-6030, US

(21) Appl. No.: 10/332,440

(22) PCT Filed: Jul. 10, 2001
(86) PCT No.: PCT/HU01/00078

(30) Foreign Application Priority Data
Jul. 10, 2000 (HU) P0002597

(51) Int. Cl. A61K 35/16

(52) U.S. Cl. 424/529

(57) ABSTRACT

Pharmaceutical preparation for the treatment and follow-up care of tumors that comprises the blood plasma or predetermined blood plasma components of equidae animals with pair number of fingers, preferably cattle being not endangered lethally by leucosis. The predetermined fraction is the material bovin 40 and/or bovin 300 constituting the difference detectable by electrophoresis between the lipid-free fraction taken from a cattle having leucosis and the lipid-free fraction taken from a healthy cattle.

In the separation method of the required fraction the initial blood is optionally treated by an anti-coagulant and the corpuscles are separated therefrom, the plasma fraction is treated by a first organic solvent, then a surfactant material composed of fine grains is added thereto, the liquid is mixed and the lipid-free fraction bound to the grains is separated from the liquid components by centrifugation, and the separated fraction is brought again into a solution.

The invention relates to a pharmaceutical preparation, primarily for the treatment and diagnosis of tumors, and to a method for separating the lipid-free fraction of blood plasma.

The published international patent application WO 00/40256 relates to the tumor inhibiting effects of the blood plasma obtained from patients suffering in acute leukemia. In the application a multiplicity of experiments is described, in which the preparation used caused a 20% improvement relative to the control group in connection with particular types of tumors. This improvement is close to the lower range of those referred to as significant.

The applicability of pharmaceutical preparations made from acute leukemic blood is limited first by the small number of the subjects that can be used as donors and by the existence of strict ethical and legal considerations in connection with the use of human blood. The outstanding significance of tumor treatment and the extent of research made worldwide in this field justifies the thorough analysis of every new way that appears as perspective either in therapy or diagnosis.

The above-referred patent application has made it likely that the blood of subjects suffering from acute leukemia comprises an exactly not defined component, which has a general anti-tumor effect.

The object of the invention is to provide a new pharmaceutical preparation that can be used for the therapy and diagnosis of tumors, including the object of the efficient separation of the blood plasma component that has a role in fighting tumors, and a further object is to find suitable groups of donors of such blood.

For attaining the objects in the first step a more efficient cleaning process has been devised, wherein it was supposed that the efficient components are in the lipid-free fraction of the blood plasma. It has been recognized that the separation of the lipid-free components of the blood plasma can be carried out in a preferable way if the plasma fraction is treated by a first organic solvent, then a surfactant is added that is composed of very fine grains, and following an intensive mixing the lipid-free fraction which has been bound to the grains is separated from the liquid component by centrifugation, then this separated fraction is brought again into a solution.

The cleaning will be more efficient if the solving and separation steps are repeated by using a second organic solvent followed by a repeated centrifugation.

The amount of the surfactant agent is about 0.5% relative to the mass of the plasma and its grain size is between about 200 to 400 mm. Preferable materials are e.g. the bolus alba, activated carbon or methocel.

The centrifugation can preferably occur with an acceleration of 15 000 g. The dissolving is facilitated if following the application of the organic solvent the solution is stored for a longer period at an elevated temperature with intermittent mixing.

The separation of the surfactant grains from the lipid-free component is not an indispensable objective, and following the second separation the grains together with the lipid-free plasma component bound thereto can be brought e.g. into a physiologic solution.

According to a further way of purification the lipid-free plasma component is brought into solution with a slight detergent, and by using a further centrifugation the solid component can be separated.

Since the lipid-free product separated in this way and obtained from donors having acute leukemia has proven significantly more efficient than the product described in the above referred patent application, the main aspect of the object was considered, i.e. whether such an anti-tumor component can be found in acute leukemic blood only, or it is present also in the blood of subjects healed from tumor.

It has been recognized that such a component is present in the blood of subjects healed from tumor, and this presence occurs in the lipid-free portion of the blood plasma. By applying the above described cleaning method it has been experimentally verified that in case of S 180 sarcoma of mice the time of survival will be substantially longer in case of using a preparation obtained from donors healed from tumor, and the effect does not appear to depend on the type of the tumor, and a lipid-free fraction obtained from the mixture of blood plasma obtained from donors healed from different tumors had a similarly efficient tumor inhibiting effect.

Although the number of donors healed from tumor is substantially higher than the number of subjects having acute leukemia, and the amount of blood that can be obtained from them is less limited, a wide range medical use cannot be expected due to the moral and legal limitations referred to above.

A further approach to solving the basic objective has lead to a further inventive discovery. The way to this discovery has come through the study of the spontaneous healing of tumors in animals. In case of equidae animals with pair number of fingers a tumor disease caused by retro virus is rather specific. In certain species the disease manifests itself in tumor, and the infected animals die, while at other species, primarily at beeves (cattle) the disease does not manifest itself in tumor or in any decrease in the general health status of the animals. The infection can be detected by the presence of the anti body GP 5.1 in the blood.

It is the recent general view of veterinary medicine that the cattle stock must be freed from individuals suffering in leucosis. This view is supported by the opinion published in the 4^{th} issue of the Journal of Hungarian Veterinarians in 1992, entitled: “the infection status of leucosis of cattle in the country and the possibilities of freeing the stock”, and this opinion was brought by the Veterinary Committee of the Hungarian Academy of Sciences. The extent of leucosis infection was found close to 50%. In a further publication the issue No 9, 1997 of the same journal comprises the paper of Dr. Telkes, Lajos “Freeing cattle from leucosis in Hungary” that emphasizes the significance of freeing the stock from leucosis. The extent of infection was found higher at larger farms, and at the time of the second article the extent of infection was about 17%.

The essence in the discovery lies in the recognition that the blood of the animals that have successfully and symptom-free fought leucosis, more particularly the lipid-free fraction of the plasma of their blood should comprise those components that have proven efficient in the experiments carried out with the blood of human donors. This discovery was followed by a great number of experiments that have confirmed this hypothesis from several sides and provided support for the existence of a tumor-inhibition effect being as efficient as it has been inconceivable since the fight against tumors has started.

The existence of the effect was confirmed also by the finding that similar blood components obtained from animals not suffering from leucosis did not have such effects.

Electrophoresis examination of the lipid-free blood plasma obtained from human and animal donors confirmed the existence of similar fractions in two ranges of molecular weight, and these fractions were missing from the plasma obtained from healthy donors. A first fraction with a molecular weight around 40000 referred to in the following as “bovin 40” and a second fraction with molecular weight falling in the range between 300000 and 350000, referred to in the following as “bovin 300” should be responsible for the tumor-inhibition effect.

In the knowledge of the solution according to the invention the general revision of the necessity of freeing the cattle stock from individuals having leucosis appears advisable.

The fractions bovin 40 and bovin 300 identified according to the invention possess excellent tumor inhibiting effect, and at the same time the detection of their presence assist in establishing the diagnosis of tumors.

The invention will now be described in connection with examples, wherein reference will be made to the accompanying drawings and the experiments. In the drawing:

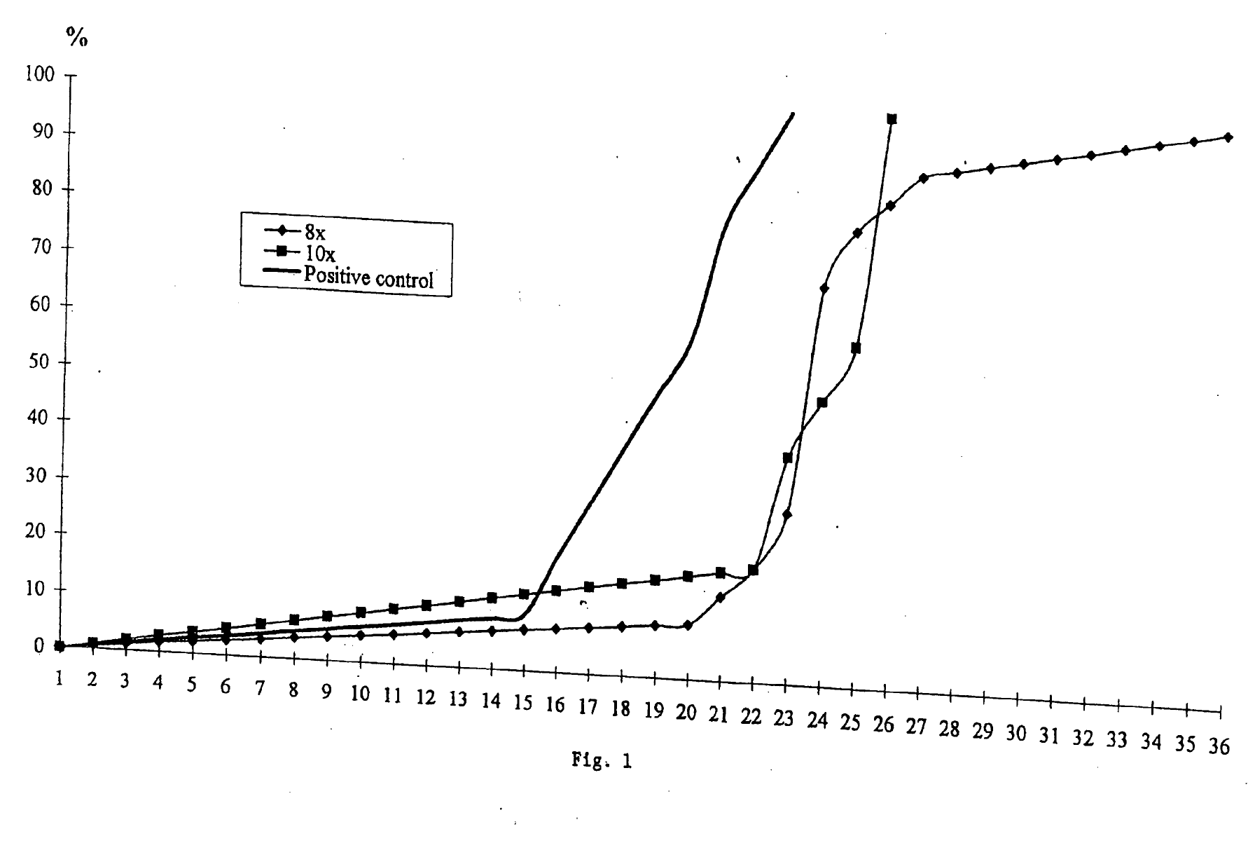

FIG. 1 is a survival diagram of a treatment with 0.15 ml Bbo-f material;

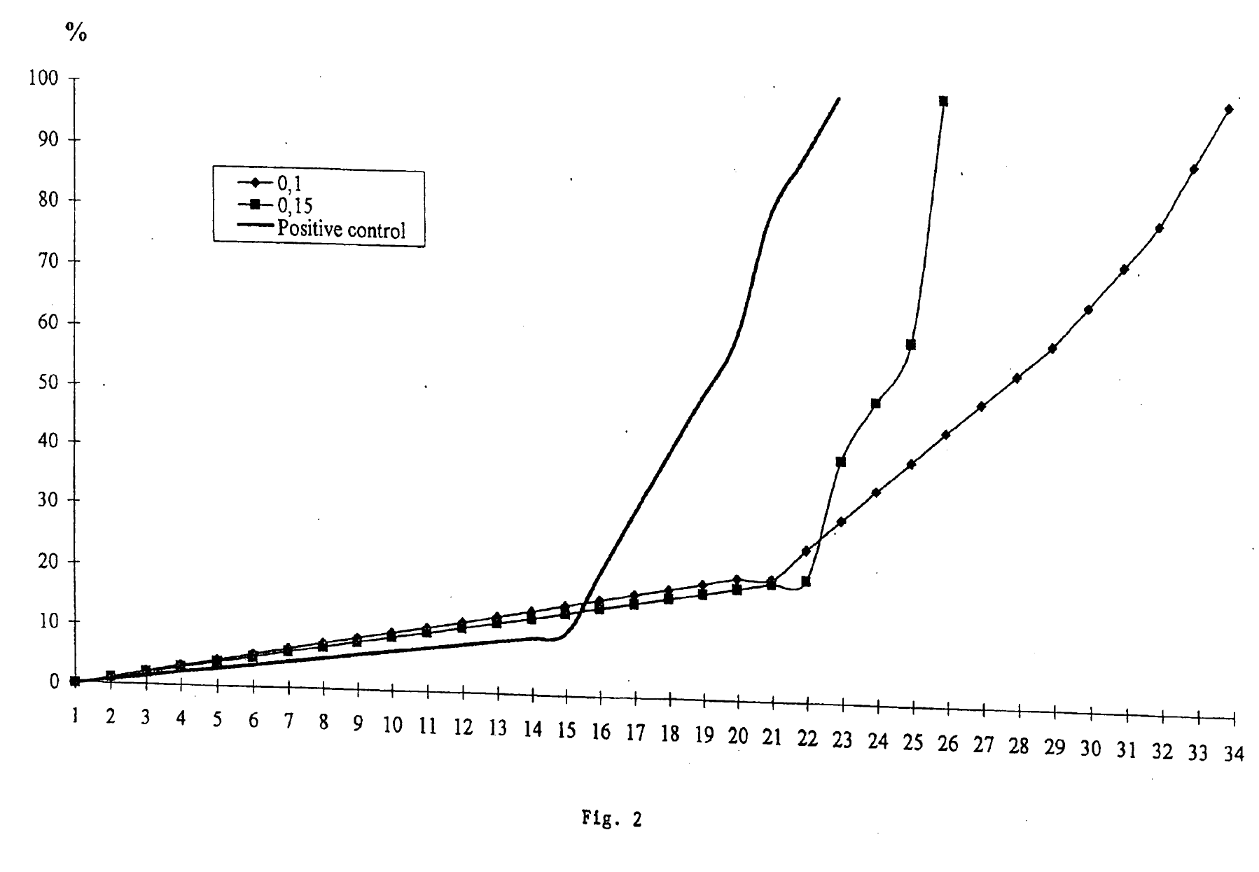

FIG. 2 is a survival diagram of 10 treatments with Bbo-f material;

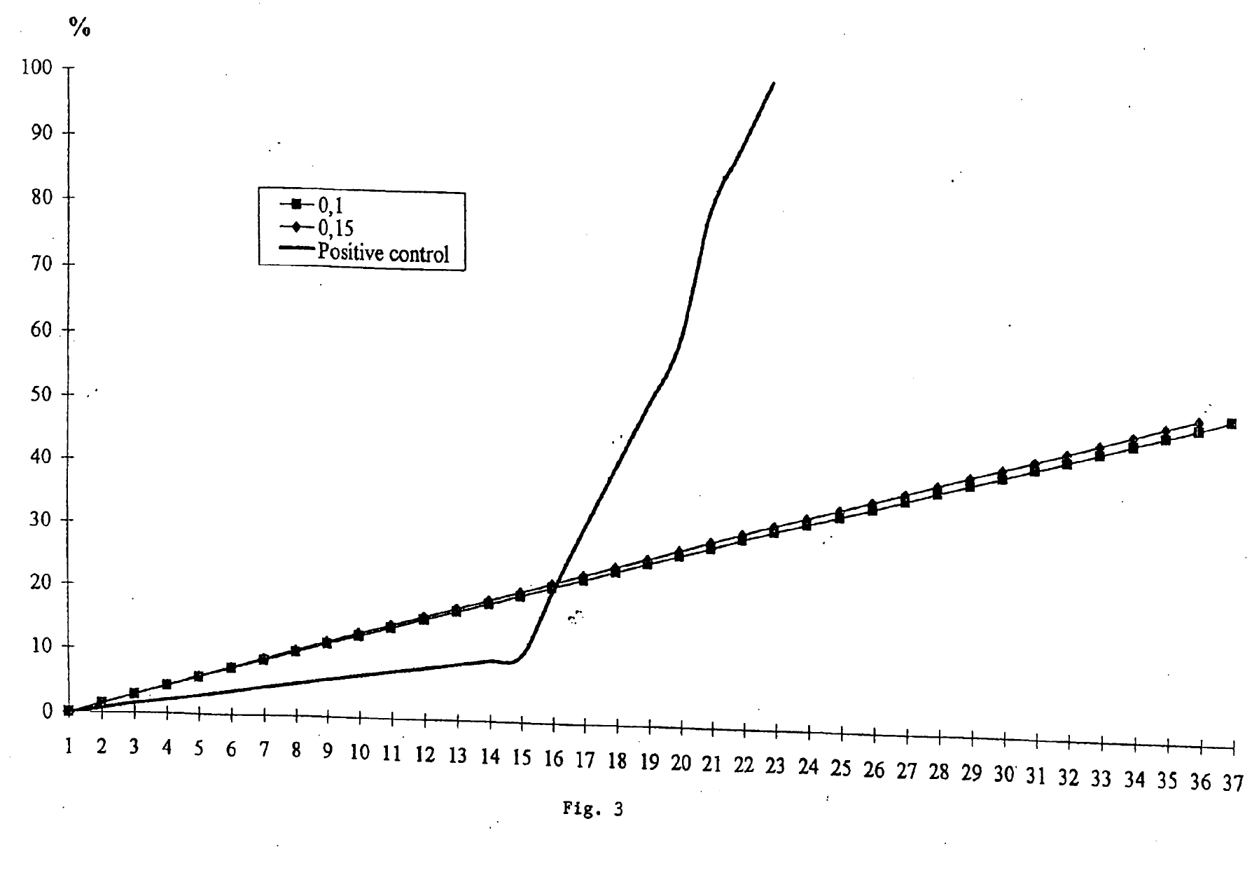

FIG. 3 is a survival diagram of 6 treatments with Bbo-b material;

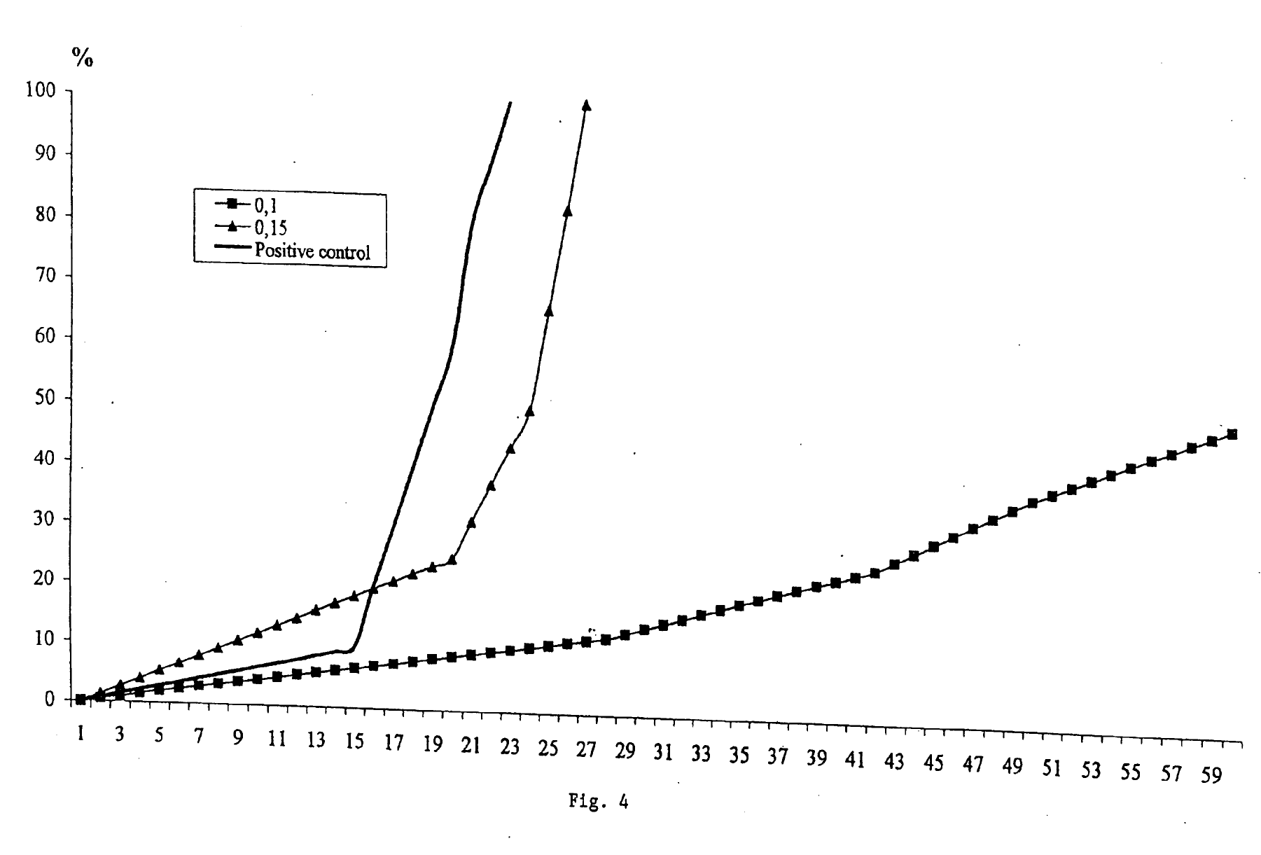

FIG. 4 is a survival diagram of 8 treatments with Bbo-b material;

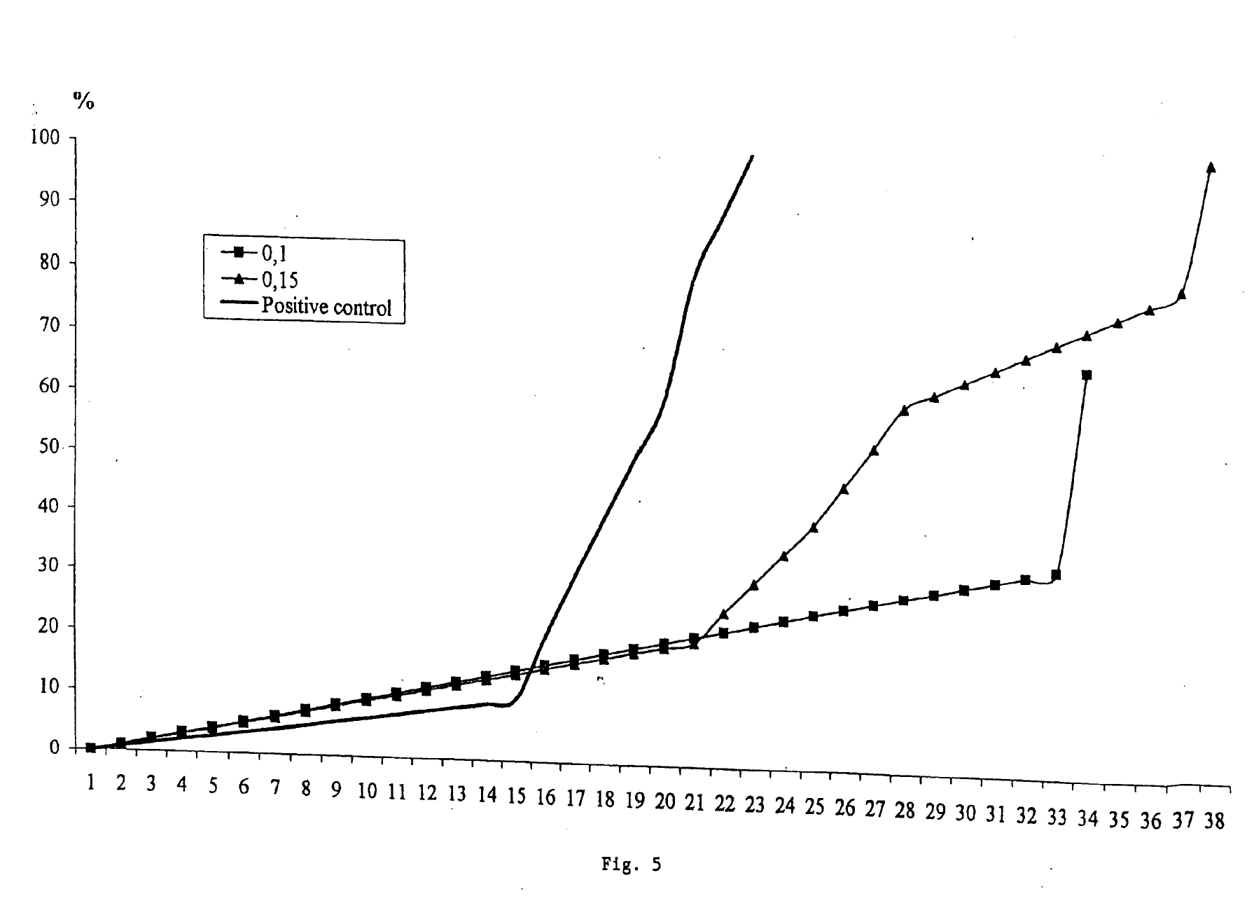

FIG. 5 is a survival diagram of 10 treatments with Bbo-b material;

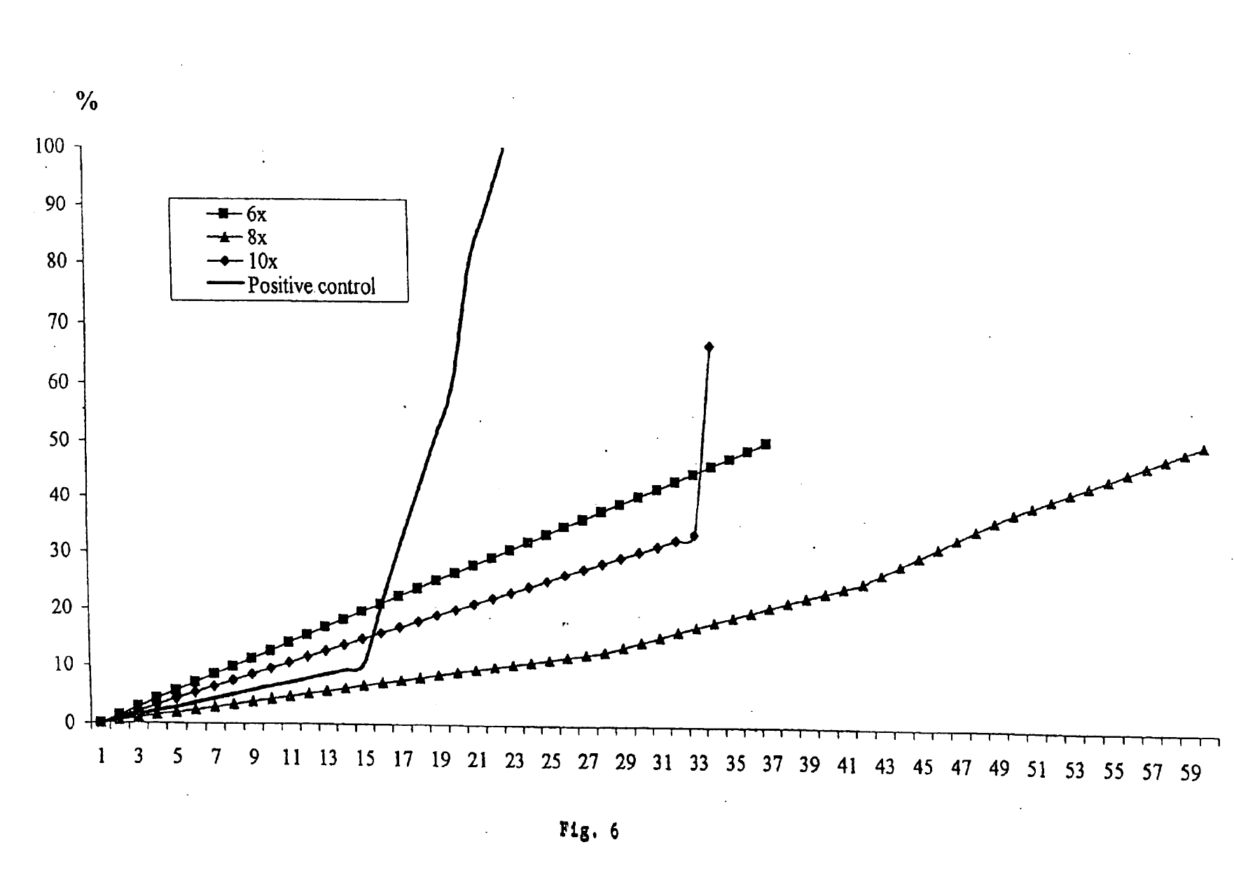

FIG. 6 is a survival diagram of a treatment with 0.1 ml Bbo-b material;

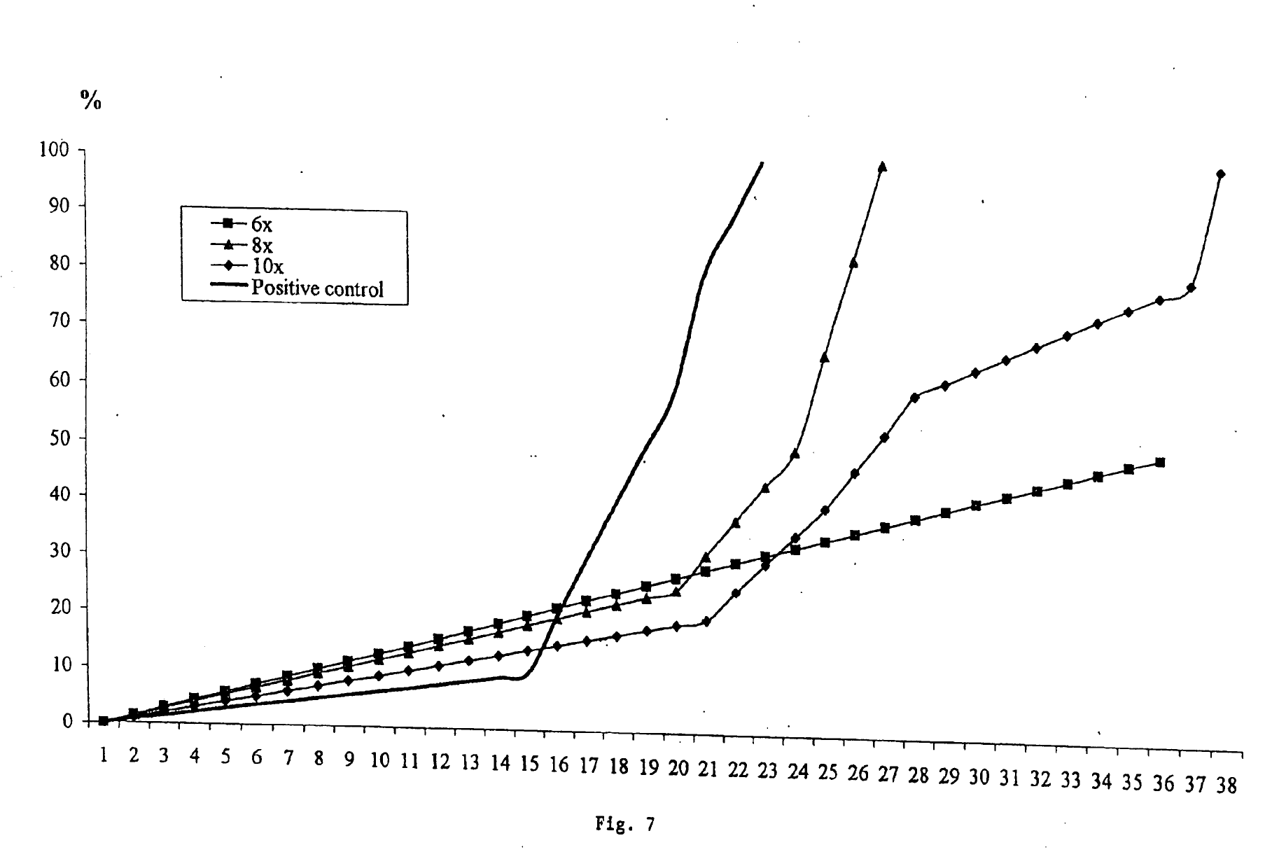

FIG. 7 is a survival diagram of a treatment with 0.15 ml Bbo-b material;

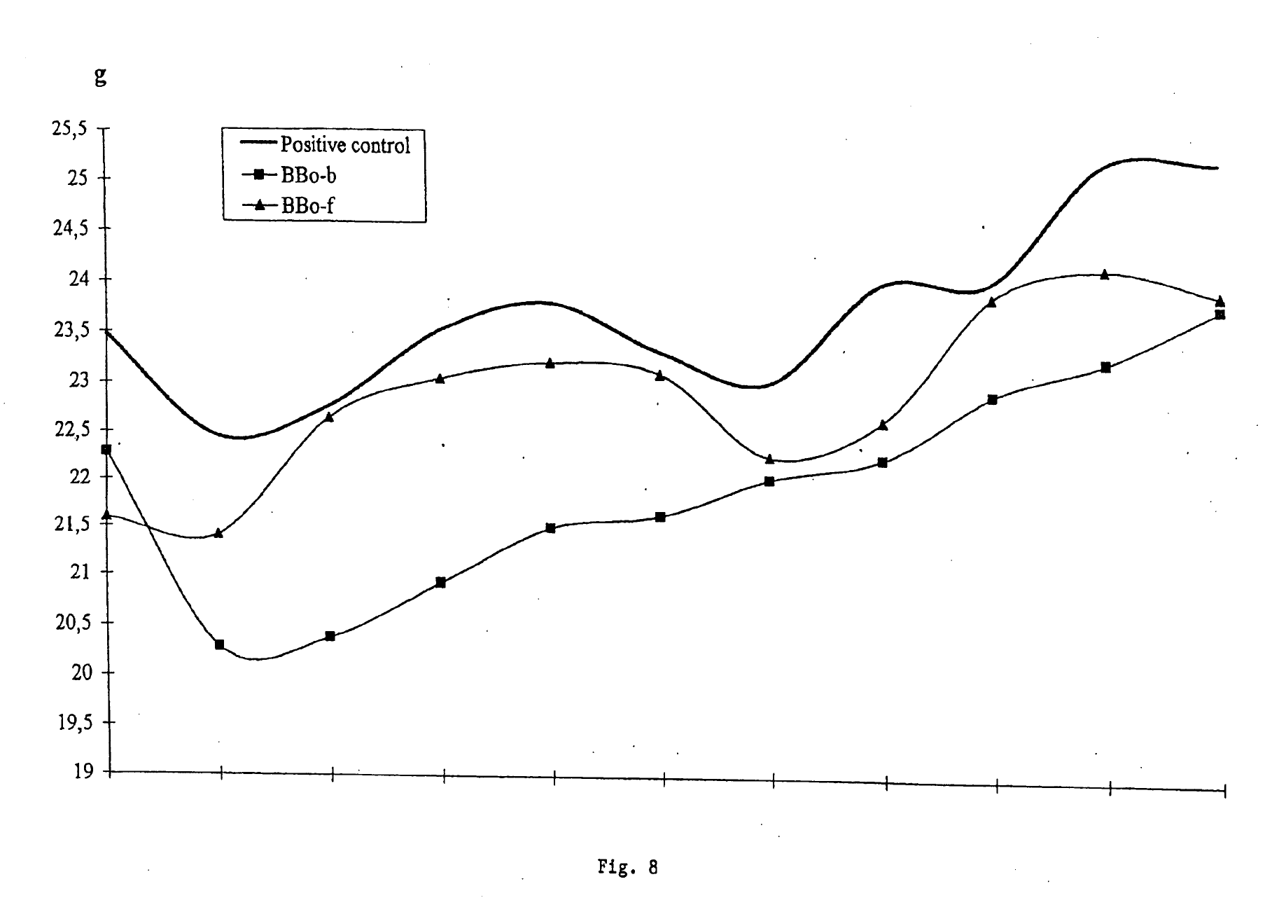

FIG. 8 shows the body weight of the animals till the 19^{th} day;

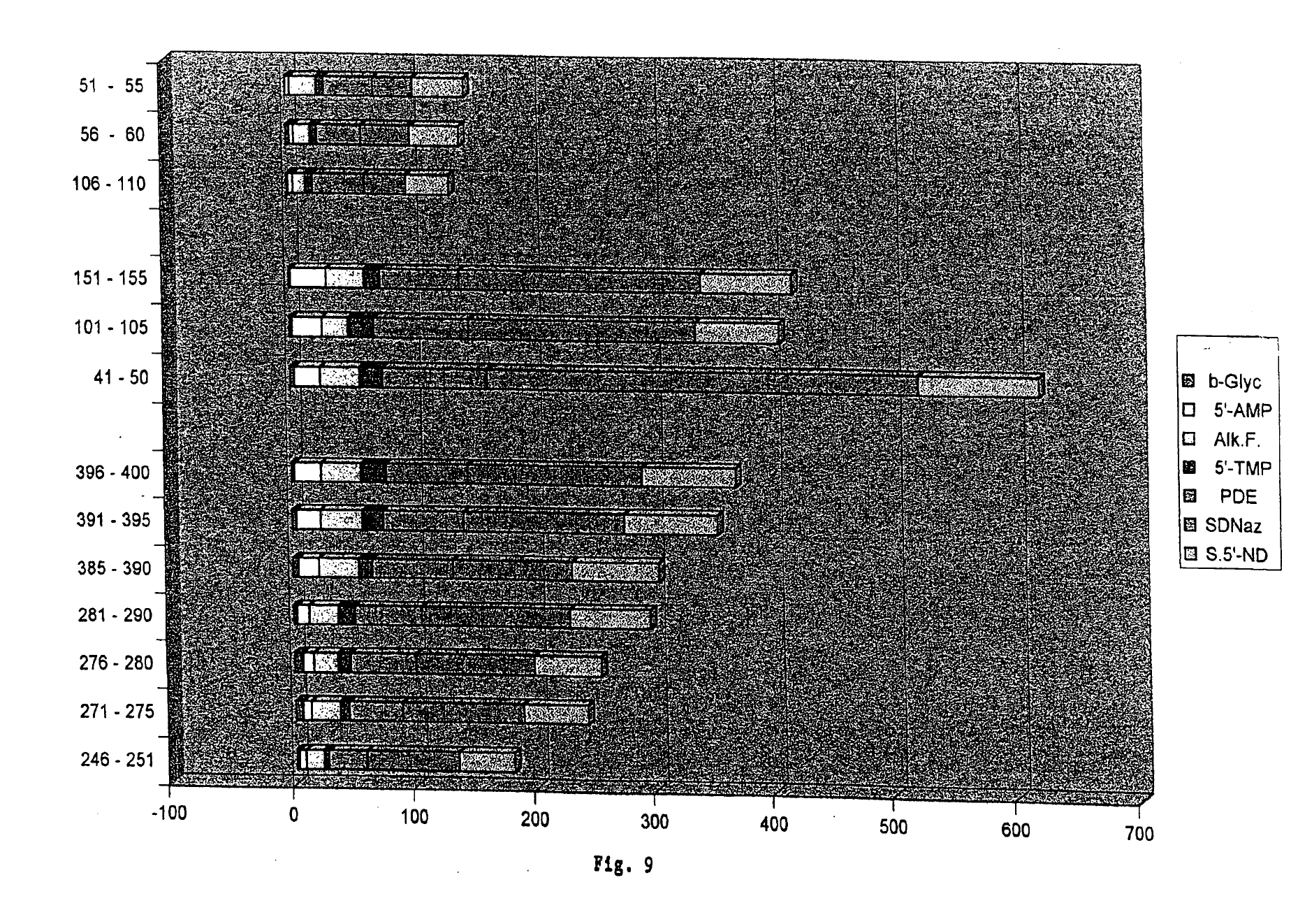

FIG. 9 is a series of column diagrams summarizing the results of enzymatic examinations;

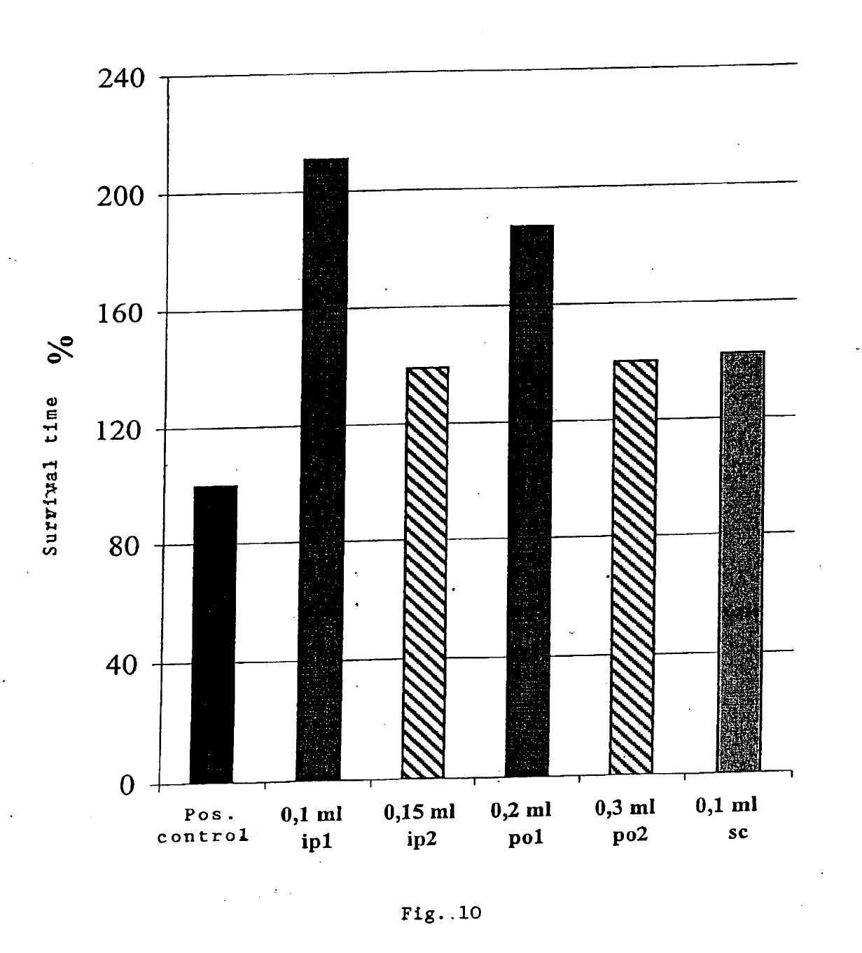

FIG. 10 shows the survival data of a treatment following the implantation of a colorectal tumor C₂₆;

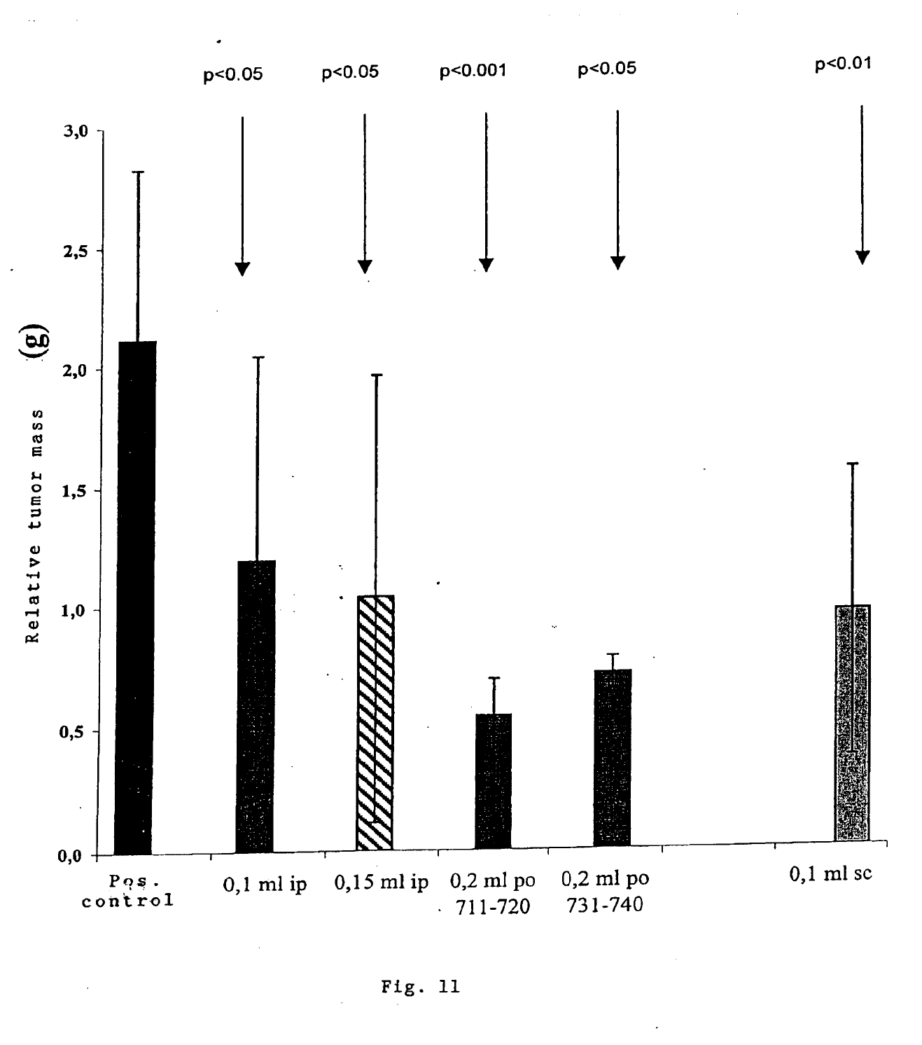

FIG. 11 shows the relative tumor masses at the treatment of FIG. 10;

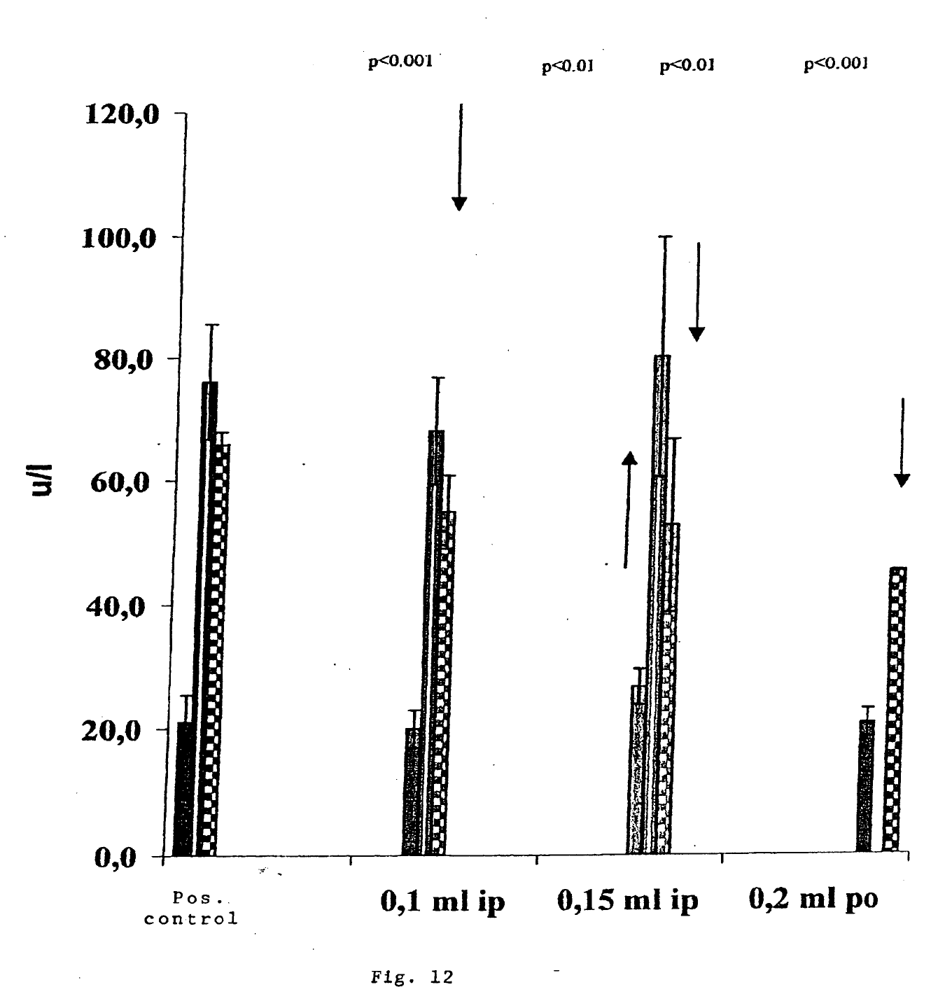

FIG. 12 shows the values of 5′ nucleotidase in case of the treatment of FIG. 10;

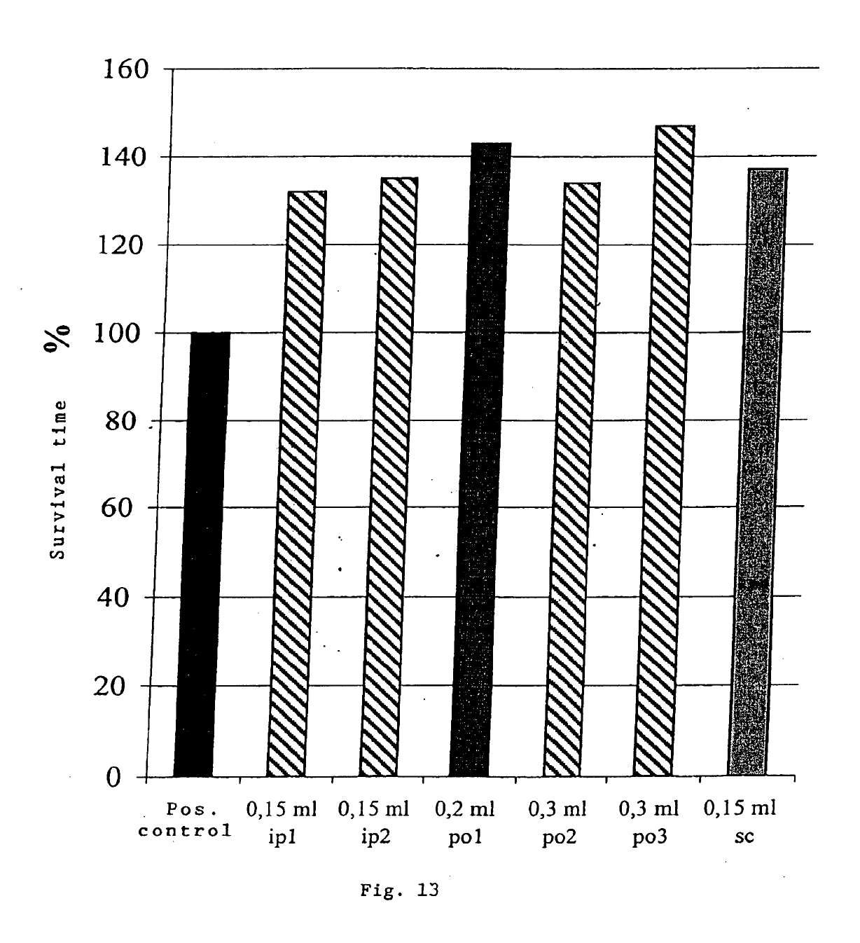

FIG. 13 shows the survival diagram experienced in case of treating MXT breast carcinoma;

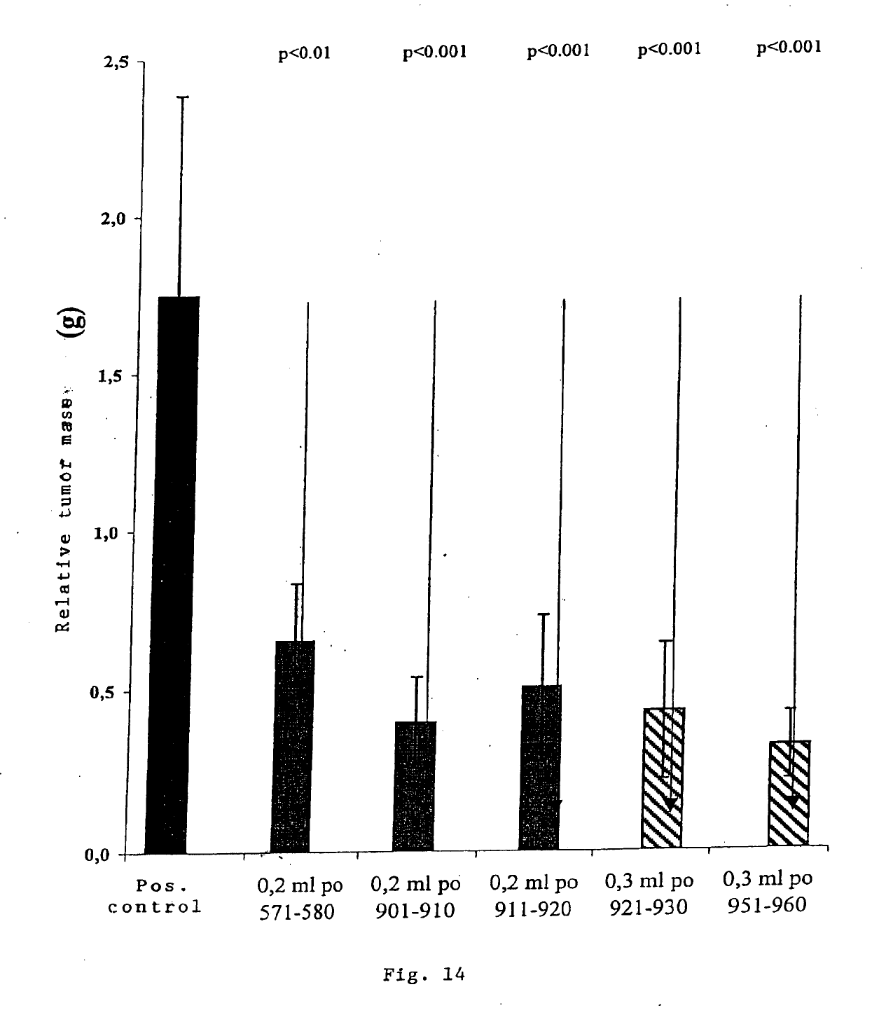

FIG. 14 illustrates the relative tumor masses in case of the treatment of FIG. 13;

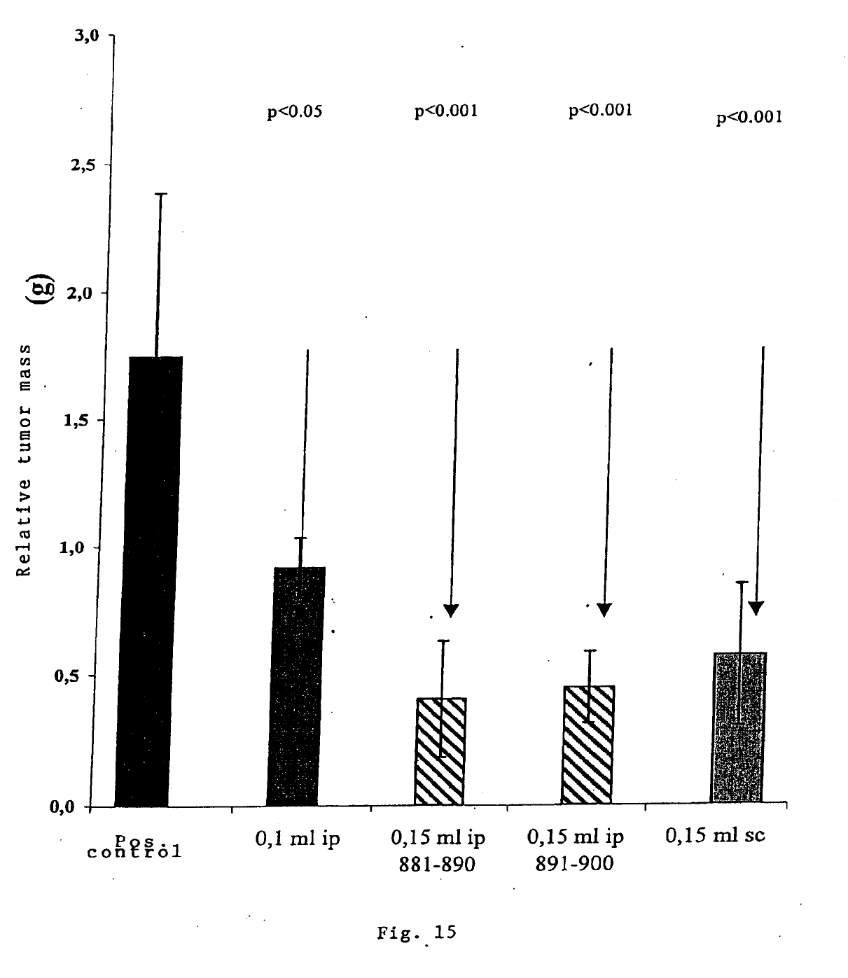

FIG. 15 is a version of FIG. 14 in case of further types of treatment;

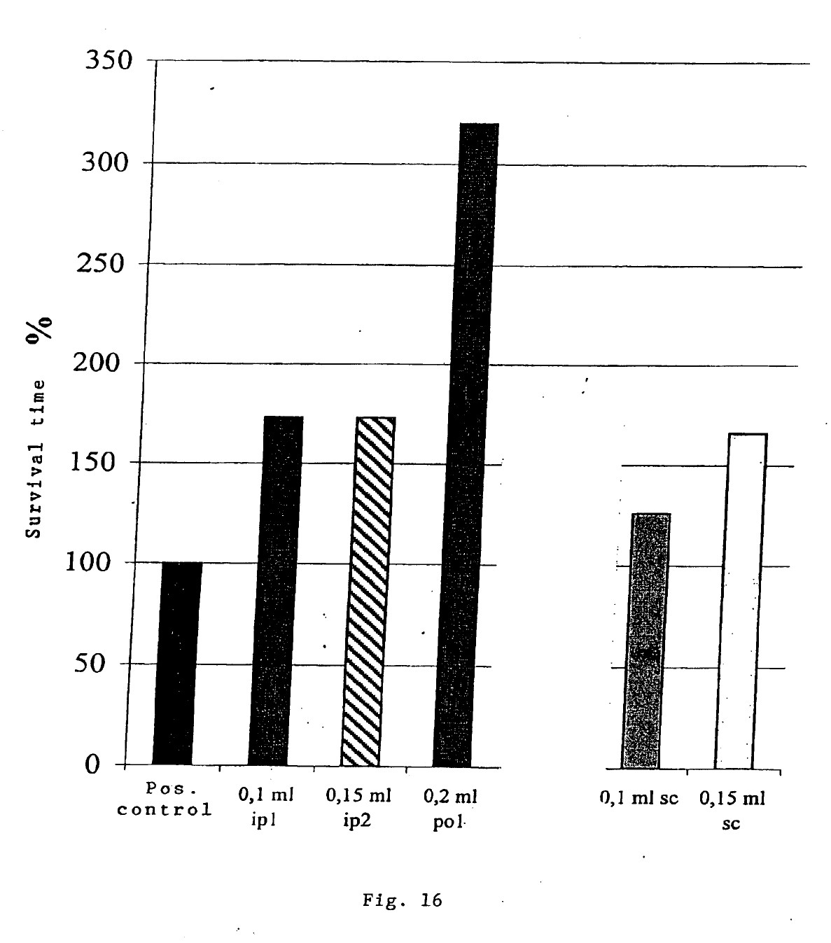

FIG. 16 shows the survival diagram experienced in case of treating L₁₂₁₀ lymphoid leukemia;

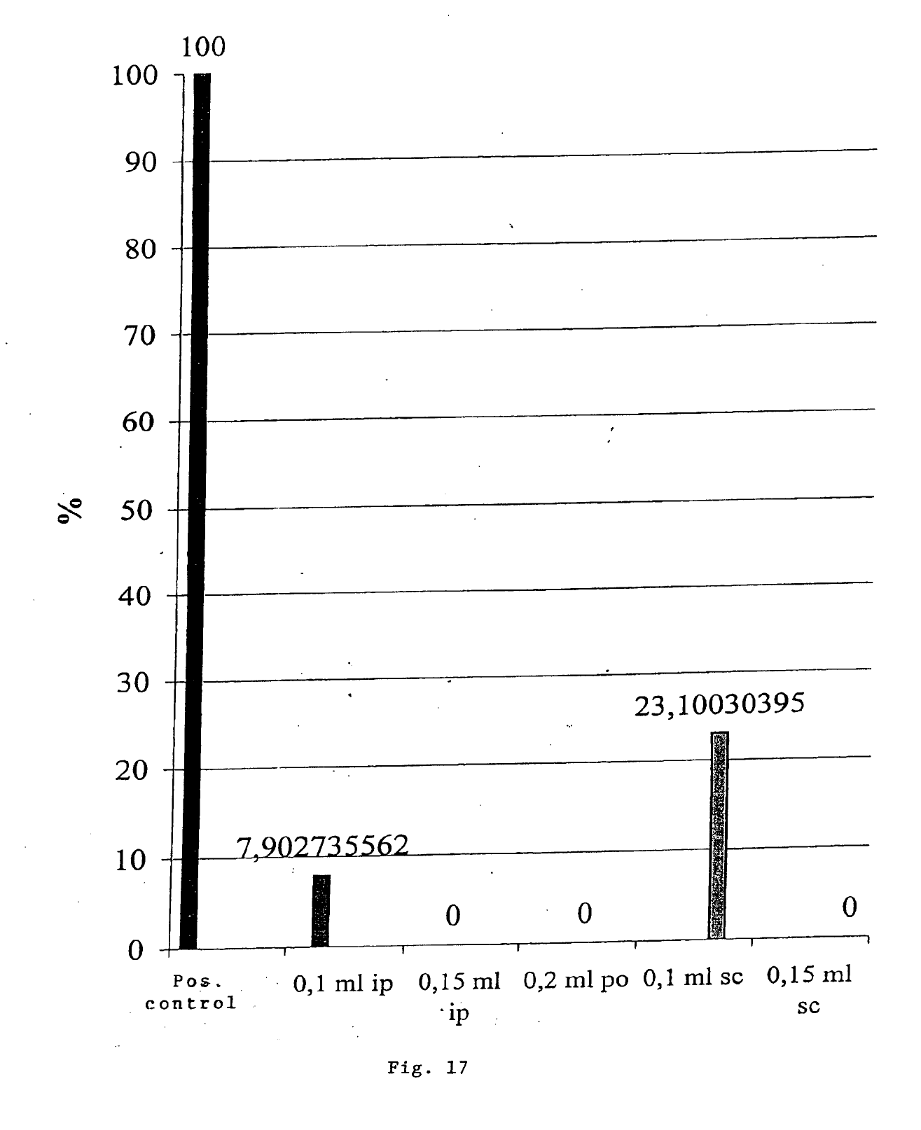

FIG. 17 shows absolute tumor mass values obtained at the treatment of FIG. 16; and

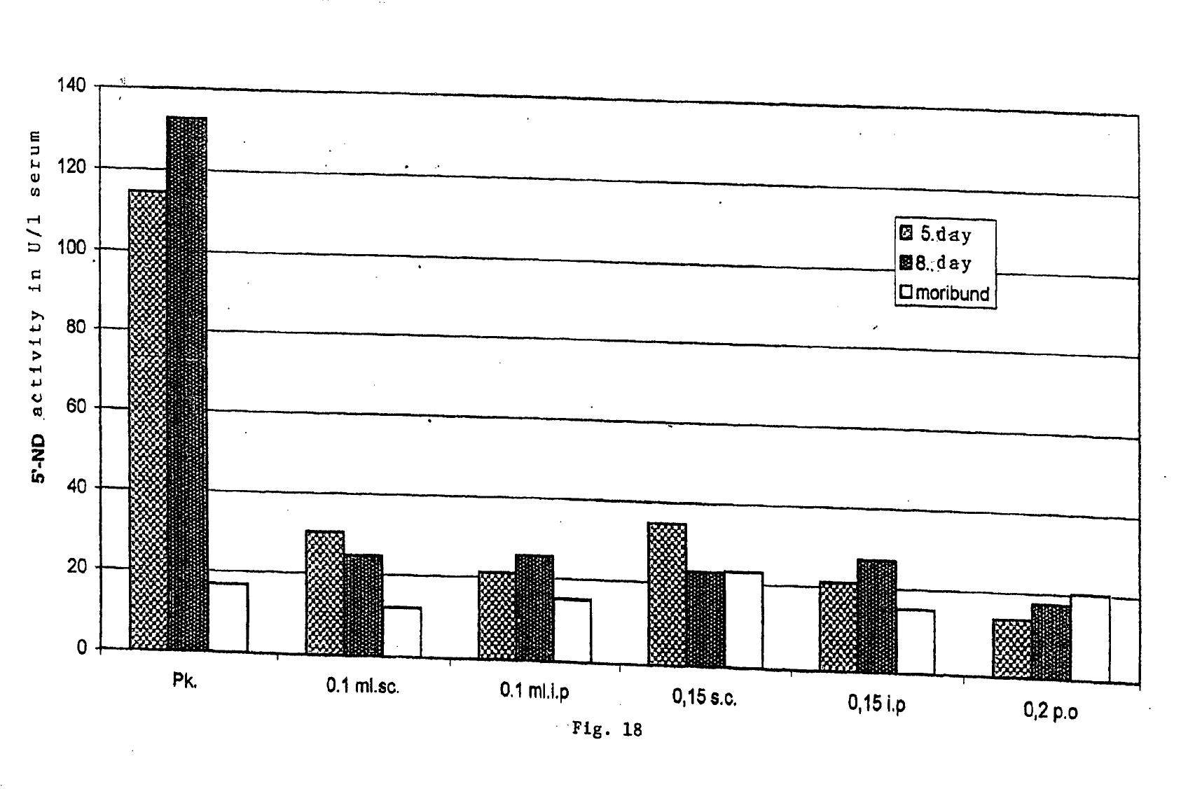

FIG. 18 shows the values of 5′ nucleotidase in case of the treatment of FIG. 16.

Different details of the solution according to the invention can be learned in the order of different stations of the experiments made. Without regard to the order of significance first the description of the preparation method of the material used for the experiments is required.

### Separating and Obtaining the Lipid-Free Plasma Component

From the blood used as starting material the lipid-free plasma components are obtained through a multi-step separation method. The preferred steps of the separation are as follows:

Shortly after the starting blood has been taken, it is treated by an anti-coagulant; being preferably heparin.

The separation of the corpuscles takes place by centrifugation, preferably at 4° C. temperature and with an acceleration of 5000 g [g: meaning acceleration of normal gravity]. In case if the centrifugation does not take place immediately following that the blood has been taken, the blood treated by the coagulant can be stored at a cooled temperature, preferably at the temperature of the centrifugation at most through 48 hours. The duration of the centrifugation is at least about 10 minutes. For the further processing the upper liquid is used. The so obtained plasma can be cooled till a temperature of −20° C. and it can be mixed with plasmas obtained similarly from different donors. The further processing can take place, when required, with a higher plasma quantity. During the examples described in the present specification the plasma obtained from any particular donor has not been mixed with those obtained from different donors, and any difference therefrom is separately reported.

As a first step of the removal of the lipid components, the plasma has been diluted by the same mass of a first organic solvent, e.g. with alcohol of 96% purity, and the so obtained solution is mixed.

In the second step a surfactant material (agent) is added to the solution in an amount of 0.5 mass %. The task of this surfactant material is to bind the lipid-free components of the plasma on its surface. The surfactant material can be bolus alba, activated carbon or methocel, and the average grain size thereof lies preferably between 200 and 400 mm. In case of the examples described the surfactant material was bolus alba.

This plasma mixture was kept permanently in a suspended state by means of physical intervention (mixing) at room temperature through 6 hours, then at a temperature of 5° C. it was incubated through 10 to 12 hours. In the period of incubation the liquid was mixed for respective short periods once in every half an hour.

Following the incubation the mixture was re-suspended by mixing, and the suspension was centrifuged at the same temperature of 4-5° C. with an acceleration of 15000 g for a period of 10 minutes.

From the centrifugate the more solid components bound to the surfactant particles were separated for further processing, and the liquid phase was disposed off. A second organic solvent was added to the separated phase in an amount equal with the mass of the first organic solvent, which in the exemplary case was the 50 mass %−50 mass % mixture of alcohol and toluene. The processing of the so obtained mixture was identical with that of the treatment after the first solvent. During this process the plasma mixture was kept permanently by physical intervention (by mixing) in suspended state through 6 hours at room temperature, then it was incubated at 5° C. through 5 to 12 hours. In the period of incubation the mixture was mixed once in every half an hour for respective short periods.

Following the incubation the mixture was resuspended by mixing, and at the same temperature of 5° C. the suspension was centrifuged through 10 minutes with an acceleration of 15000 g.

From the centrifugate the solid components bound to the surfactant particles were separated for further processing, and the liquid phase was disposed off.

The solid component was spread out to form a thin layer, then any remaining organic solvent was removed in a dryer placed under a vacuum for two hours.

Then a physiologic saline solution with a mass equal to the starting plasma mass was added to the separated sediment, and the material was resuspended by mixing. The so obtained plasma preparations will be labeled in the following part of the specification with the letter “b” that refers to the initial of the surfactant material being bolus alba.

In obtaining a second, relatively more refined alternative plasma preparation the surfactant material was removed in such a way that a tissue-friendly detergent was added to the mixture “b” that is capable of dissolving the plasma preparation. In the exemplary case this detergent was 0.01 mass % of sodium lauryl sulfate. For the sake of obtaining an appropriate suspension, the material together with the detergent added thereto was incubated at room temperature through 6 hours under continuous mixing then it was stored in a refrigerator through 8 to 12 hours. The material that formed a sediment during incubation was resuspended, then in a cooled state it was centrifuged with an acceleration of 15000 g. The sediment was disposed off, and the upper liquid comprised the useful material, which will be labeled in the following part of the specification with the letter “f” for distinction from the material “b” and designating that this material is finer.

For facilitating the experiments carried out with mice both types of products were fed in respective 1 ml doses, they were then labeled and stored in a freeze state.

The products and the circumstances of their preparation were both sterile, therefore the material was aseptic and capable of parenteral applications.

### 1. Experiments with Implanting S 180 Sarcoma

All these experiment were carried out with identical type female BDF₁ mice with an average weight of 25 g. Both in the examined and in the positive control animals S 180 sarcoma was transplanted in a subcutane manner. The circumstances of the experiments regarding the type of the animals, the type of the implanted sarcoma and the way of implantation were identical with those described in the earlier referred international patent application. Each experimental group comprised at least five mice, and the data reported relate to the average of the mice in the associated group. The mice were distinguished according to appropriate groups of numbers and they had individual numbers.

Experiment 1

From the blood of a patient suffering in acute leukemia a plasma preparation of “b” type was prepared, and in the experimental group every mouse obtained subcutane 0.1 ml therefrom in every other day, altogether at eight times.

In the positive control group the average life time was 19.6 days, and in the experimental group it was 23.5 days. This is a 20% increase that can be regarded as a significant improvement. In the above referred international application in connection with the same type of tumor the best preparation could result in a 5% increase in survival, which was still below the significance threshold. The appropriate separation of the lipid-free plasma component is therefore very essential.

Experiment 2

This experiment comprises the results of six independent experiments, and it is based on the supposition that the material efficient against tumors which is supposed to be comprised in the lipid-free component of the plasma is present also in the blood of subjects healed from a tumor. Such persons were selected as donors, who has been alive at least for 10 and at most for 27 years since their tumor had been discovered, therefore they can be regarded as healed. The diagnosed tumor type of the donors were in sequence larynx, colon, breast, lymphoid leukemia, testicle and prostate as well as lung tumors.

The plasma preparation type “b” was made in case of all the six donors, and respective groups of mice were treated therewith. The average survival time of the control group was again 19.6 days, however, the average of the treated groups varied between 25.5 and 27.3 days, there was therefore only a slight difference between the treated groups. Such a survival constituted an improvement between 30% and 40% relative to the control group, which is very significant.

As a further experiment a “b” type plasma product was made from the equal mixture of the plasmas obtained from all of the six donors after the removal of the corpuscles. This product provided an increase in survival by 38% relative to the control group.

These experiments have thus confirmed the hypothesis according to which the lipid free plasma component of subjects healed from a tumor includes an efficient tumor-inhibiting material. Furthermore it was also proven that the existence of such a material does not depend (at least in case of the examined tumor type) on the actual type of the tumor, therefore the mixture is equally efficient.

Experiment 3

Such cattle (beeves) were chosen as donors, at which the blood examination confirmed the existence of cattle leucosis. From the blood of such donors “b” and “f” type of plasma products were made. Similarly, both types of plasma products were prepared from the blood of cattle, whose blood did not show the presence of leucosis.

The examinations were carried out in several versions, wherein as variable the first parameter was the number of treatments applied every other day, the second parameter was the amount of dose which changed between 0.1 and 0.15 ml, and the third parameter was the purity of the product i.e. being either “b” or “f” type. The preferred range of examination of these parameters were determined by pilot experiments preceding the actual experiments, since respective optimums were found regarding both the dose and the number of treatments.

The product obtained from the blood of cattle having leucosis satisfies the criteria of large scale applicability, since there are no limits regarding the availability of donors and regarding ethical or moral considerations. The survival data substantially exceed the otherwise favorable data of the preparations obtained from human blood. This is the reason of the detailed description of the results of these experiments.

During the experiments the average weight of the animals, the average survival time were examined, furthermore at predetermined phases of the experiments blood samples of 1 ml were taken from respective mice of each group, and in the samples the presence of a plurality of tumor markers was examined. The mice from which blood samples were taken, were removed from the following examinations, since they could not survive giving such an amount of blood.

### Data Relating to the Survival Time

The results relating to the survival time can be divided in two main groups, namely whether the treatment took place with “b” or “f” type of product. The origin from cattle having leucosis is designated by the abbreviation “Bbo”, therefore the treatment of the first main group took place with the material Bbo-b and that of the second main group took place with the material Bbo-f.

The days are counted from the implantation of the tumor. Each curve shown in FIGS. 1 to 7 relate to the average of a group including at least five mice.

FIGS. 1 and 2 show the survival diagrams of groups treated with the material Bbo-f. The mice in the positive control group did not obtain any treatment following the implantation apart from normal feeding, the mice in the treated group were treated every second day by the material Bbo-f having the defined mass. In each treatment at the groups shown in FIG. 1 the dose was 0.15 mm. The two treated groups differ from each other in the number of treatments. In the first group 8 treatments were applied, while in the second one the number of treatments was 10, thereafter the mice did not obtain any further treatment. In case of the group obtained 10 treatments it can be seen that following the termination of the treatment, i.e. after the 20^{th} day the mortality rate has suddenly increased. This sudden increase can be seen also at the group that has obtained 8 treatments, but it is interesting to see that the increase takes place later and with slighter slope. The average survival time relative to the control group with 19.6 days is at the group that has obtained 8 treatments is 24.8 days while at the group that has obtained 10 treatments is 23.2 days.

In each of the groups shown in FIG. 2 the treatment took place 10 times on every second day. The difference lies in the dose. The mice in the first group obtained 0.15 ml dose at each treatment, while that of the second group were treated with a dose of 0.1 ml. The difference is very apparent when the dose was decreased. The survival time increased to 30 days which constitutes a 153% improvement relative to the positive control group.

The results of the treatments with the material Bbo-b are shown in 2 groups of Figures. In case of FIGS. 3 to 5 the changing parameter is the dose. In the case shown in FIG. 3 the treatment took place 6 times, and it can be seen that the survival time hardly depends on the dose used, the lower dose is slightly better. The survival time was, however, substantially better compared to the material Bbo-f, since the average survival time was 37 days which is a nearly 189% improvement relative to the positive control. The dependence of the survival time from the dose will be more apparent when FIG. 4 is considered. Here the number of treatments was 8. Under the effect of the dose of 0.15 ml the survival time has decreased compared to the value obtained at 6 treatments, and this could be only the consequence of an overdose. In contrary the dose of 0.1 ml appears to be optimum with 8 treatments, since the average survival time has increased substantially, and by the 59^{th} day the diagram has not yet reached a 50% value. By this the survival has increased over 300% which is an outstandingly favorable figure.

FIG. 5 shows the average survival time in case of mice obtained 10 treatments. Surprising here the property of the 0.15 ml dose, because in this group the survival decreases suddenly but the average is better then at the earlier group with a similar treatment. Owing to the low number of mice the differences could come from the more ore less favorable behavior of one or two mice. The sudden mortality increases in case of dose of 0.1 ml after the 33^{rd} day which might also be the consequence of an overdose.

In FIGS. 6 and 7 the variable parameter is the number of treatments. The comparison of the two figures demonstrates here also the application of 8 treatments with a dose 0.1 ml as optimum. From FIG. 7 the consequence can be drawn that in case of higher dose the improvement increased with decreasing number of treatments, which also indicates that the dose was too high.

### The Healing of the Tumor and the Average Weight of the Animals

The improvement experienced in the status of the animals of the treated groups can be recognized not only on the basis of the average survival time. In the animals with starting weight of 25 g such a large tumor is formed which weights 6 to 8 g i.e. nearly a third of the full weight of the animal. In the animals treated with 0.1 ml dose from the material Bbo-b eight times by the end of the cycle of treatment a well recognizable has taken place in the status of the tumor, the sarcoma opened and a squashy material was discharged. This material comprised dead tumor cells. The “hump” of the animals disappeared and they started taking up weight.

FIG. 8 shows the value of the average body weight of the animals in the first 19 days regarding the groups Bbo-b and Bbo-f, both obtained 8 treatments and the positive control group. The diagrams is in good correspondence with the above described status report, especially in case of the treatment with the material Bbo-b. The drawback of the diagram lies in that it does not show separately the mass of the tumor. We could not obtain an accurate value for the tumor weight, therefore the full body weight also includes the weight of the tumor. In the treated group the initial decrease followed by a regenerative increase is typical, in case of the positive control group the weight increases in a fluctuating manner caused mainly by the increase of the tumor.

Of the above experiments the ones appeared as bests were repeated with such a material that was obtained from healthy cattle that did not have leucosis. The results did not significantly differ from those obtained at the positive control group, and this has confirmed that only the material obtained from the blood of cattle having leucosis is effective.

### The Results of Enzymatic Tumor Marker Examinations

For the examination of metabolic changes accompanying malignant processes there are several enzymatic tumor markers which are widely determined in the clinical practice, and of these methods a number of types were chosen which can be used for routine examinations. In selecting the methods it has been taken into account that malignant processes are accompanied and often maintained by complex biochemical disturbances. Therefore, due to these considerations it seemed necessary to carry out assays of nucleic acid metabolism (serum alkalic(basic) and acidic dezoxyribonuclease activity, 5′-nucleotidase activity); of polyamine metabolism (arginase activity); of the function of liver mithochondria (ornithine carbamoyl transferase activity); of gluconeogenesis (phosphohexose izomerase activity); and of identifying bone-related processes (bone-specific alkalic phosphatase activity). The changes in the activity of the listed enzymes follow the metabolic changes accompanying and maintaining the malignant processes and the results of the applied therapies.

During the experiments blood samples were taken from the mice on the 8^{th}, 15^{th} and 19^{th} days following the implantation of the tumor. The data are summarized in Table 1. The data relate to the average of respective five mice, all examined separately.

The meaning of certain columns in the Table:

β-Glyc: non-specific phosphatase, β-glycerophosphate; this value should be subtracted from the value of the other phosphatase values in order that they can be interpreted as tumor markers;

5′-AMP: 5′-nucleotidase enzyme family, more particularly: 5′-adenosine-5-monophosphate

Alk. F: alkalic phosphatase

5′-TMP: 5′-thymidine-5-monophosphate

PDE: phosphodiestherase

SDNaz: acidic dezoxyribonuclease (acidic DNAse)

Σ.5′-ND: the total value of all 5′-nucleotidase

TABLE 1
The effects of plasma serum Bbo-b and Bbo-f on mice having S-180
sarcoma

| Animal groups | β-Glyc | 5′-AMP | Alk.F. | 5′-TMP | PDE | SDNaz | Σ.5′-ND |
|---|---|---|---|---|---|---|---|
| 246-251 | 2.12 | 5.45 | 14.7 | 4.67 | 31.2 | | 45.7 |
| 271-275 | 5.76 | 7.12 | 23.5 | 7.89 | 44.6 | 100.7 | 53.4 |
| 276-280 | 6.78 | 8.92 | 19.9 | 10.78 | 54.6 | 98.3 | 55.67 |
| 281-290 | 2.67 | 10.23 | 23.9 | 13.6 | 55.9 | 123.1 | 67.3 |
| 385-390 | 4.56 | 16.8 | 32.8 | 11.7 | 67.2 | 98.9 | 72.4 |
| 391-395 | 3.56 | 19.9 | 34.7 | 17.3 | 66.8 | 134 | 77.9 |
| 396-400 | 2.45 | 21.8 | 33.5 | 20.76 | 67.9 | 145 | 78.5 |
| 41-50 | 3.33 | 21.3 | 32.78 | 19.43 | 86.8 | 359.1 | 97.8 |
| 101-105 | 2 | 24.7 | 21.72 | 21.45 | 78.9 | 189.1 | 69.9 |
| 151-155 | 1.56 | 29.4 | 31.32 | 13.42 | 65.7 | 201.6 | 76.1 |
| 106-110 | 1.15 | 3.67 | 10.39 | 5.67 | 43.2 | 35.2 | 35.2 |
| 56-60 | 2.91 | 3.4 | 13.6 | 5.8 | 36.8 | 40.8 | 40.3 |
| 51-55 | −0.03 | 3.4 | 22.31 | 6.2 | 41.3 | 32.9 | 42.1 |

In the table three different groups can be seen. The last group relates to the control with mice that have not been implanted with tumor, and mice 51-55 in this group were examined on the 8^{th} day, the mice 56-60 were examined on the 15^{th} day and the mice 106-110 were examined on the 19^{th} day.

The middle group relates to the positive control with mice 151-155 being examined on the 8^{th} day, mice 101-105 on the 15^{th} day and the mice 41-50 on the 19^{th} day.

The first group can be divided in two sub-groups, the ones starting with the digit 2 obtained treatment with the material Bbo-b and the ones starting with the digit 3 were treated with the material Bbo-f. In the sub-group Bbo-b the examination of mice 281-290 took place on the 8^{th} day, of mice 276-280 on the 15^{th} day of mice 271-275 on the 19^{th} day and finally the examination of mice 246-250 took place with regard to the substantial survival much later, on the 45^{th} day.

In the second subgroup with mice treated with the material Bbo-f the distribution according the day of examination: 396-400: 8^{th} day; 391-395: 15^{th} day; 385-390: 19^{th} day.

The results are summarized in the form of column diagrams in FIG. 9, in which the groups of mice illustrated on the horizontal axis were associated with data on the vertical axis above each other, wherein the height of the columns expresses the sum of the data. At the horizontal axis the mice groups were illustrated separately, and within each group the left-to-right order follows the increasing order of the sampling dates.

The three left columns are associated with the control group, the middle three columns are associated with the positive control group, and the first three columns of the seven right columns belong to the sub-group Bbo-f and the last four columns belong to the sub-group Bbo-b.

In the control group the data naturally do not change in time, their fluctuation is within the natural range. In case of the positive control group all components are extremely high, and by the moribund period (last column) they had a further increase.

On the contrary, in both treated sub-groups the initial values are already smaller than the data of the positive control group and they further decreased in time. The material Bbo-b provided in accordance with the favorable survival data substantially better results than the treatment with the material Bbo-f. The decrease continues after the 19^{th} day, and by the end of the experiment the data are close to the normal values of the control group.

Experiment 4

Based on the favorable results obtained during the previous experiments it was examined what components of the lipid-free plasma were responsible for the effect. Since we had healthy human blood, blood from subjects with acute leukemia, human blood from subjects healed from tumor, furthermore blood taken from cattle with and without leucosis, the preparation type “b” was made from each of the listed bloods and they were undertaken to respective electrophoresis examinations. For the examinations the starting material was brought on the surface of a used polyacrylamide gel, and under the effect of the electrophoresis treatment the components of the material were separated according to the order of their molecular weights.

The fractions taken from blood which has proven inefficient from the point of view of tumor treatment, i.e. normal human blood and blood taken from leucosis-free cattle was compared with fractions obtained from blood types being efficient from the point of view of tumor treatment. The most apparent difference was experienced in the fractions of blood taken from cattle having leucosis, and it lied in the presence of a fraction with molecular weight about 40000 and a further fraction with a molecular weight between 300000 and 350000. These two fractions together represented 8 to 12% mass of the sample. The fractions of the blood preparation taken from donors with healed tumor comprised both of these components, however, the presence of the fraction around the molecular weight 40000 was more apparent, and the amount of the fractions was substantially lower, about 2-3%. Among the fractions of the preparation taken from acute leukemic blood the component with molecular weight 40000 was present only in traces, the other fraction was not detectable.

For the sake of identifying said two fractions the one with molecular weight around 40000 will be referred to as “bovin 40” and the other fraction between 300000 and 350000 will be designated as “bovin 300”. The method described here is sufficient for the unmistakable identification of these two fractions. The structural identification of these fractions is currently under way. In the knowledge of the results shown it can be stated with good grounds that the presence of the materials bovin 40 and bovin 300 is responsible for the demonstrated effects.

### II. Experiment with the Implantation of C₂₆ Colorectal Tumor

The experiments were carried out with first generation internally bred Balb/c male mice.

The place of origin of the mice was the TNO Institute, Rijkswijk, The Netherlands, their place of breeding was the National Institute of Oncology (Hungary), Department of Experimental Pharmacology.

The circumstances of keeping the animals were: cage of macrolon material, temperature 20-22° C., relative humidity 45-55%, dark and light keeping changed in 12 hour's periods. The feeding occurred with standard quality mouse feed that can be sterilized in an autoclave (type: Altromin, Germany), the bedding was made of wood shavings. The hyygienic level of the breeding was in accordance with the prescribed SPF (Specified Pathogen Free) conditions. The care and keeping of the animals took place in accordance with the Helsinki declaration on “Guiding Principles for the Care and Use of Animals”.

The Colon-26 colon adenocarcinoma was taken from SRI, Birmingham, Ala., U.S.A. The way of transplantation: from the maintained tumor a piece of 20 mg was implanted subcutane into the interscapular region.

During the experiments the animals were divided into groups of ten mice, the animals in the positive control group did not obtain any treatment following the implantation. In the treated groups the treatment took place with the previously described Bbo-b material. This material was referred to during the documentation of the experiments also as ABB-7. The experiences obtained through the experiment series I. were utilized, and the treatment was applied once a day at the same time, between day 1 and 9, altogether eight times. The differences between the respective groups were in the way of the treatment and in the amount of experimental material applied.

The survival data are summarized in FIG. 10. In case of the positive control group the 100% survival time was 19 days. In the first group the treatment occurred intra-peritonial (Ipl) with a dose of 0.1 ml, in the second group the way of application was the same but the dose was 0.15 ml, and the application of the material through the mouth (per os) was also tried out. Here an appropriate dosage feeder was used to bring the material directly into the stomach of the animals. In the first per os (po1) group a dose was 0.2 ml material and in the second per os (po2) group the dose was 0.3 ml. A further group sc was also treated, where the material was applied in a subcutane (under the skin) manner with 0.1 ml material. The survival data show the averages of the groups of ten mice. From the figure it an be seen that in case of each treated group a substantial improvement took place, the most efficient two treatments were the ip application with 0.1 ml and the po treatment with 0.2 ml material.

The experiments were repeated in further groups of ten mice, and for measuring the tumor mass the tumors were removed when the animals reached their moribund states (i.e. directly preceding death) and the tumor masses were measured.

FIG. 11 shows the results of these experiments. Above the columns the values of the associated confidence levels are given, which were in all cases less than 0.05 i.e. the data are very reliable.

In further groups of ten mice treated similarly, the 5′-nucleotidase activities were determined on samples taken on the 8^{th}, 16^{th} days from the tumor implantation as well as in moribund state as in case at the experiment series I. These data are shown in FIG. 12. The activity has increased from the initial low value by the 16^{th} day to a very high one, then when the moribund state has been reached, the activity has substantially decreased from the values obtained at the positive control group.

### III. Experiments with Implanting MXT Breast Tumor

Under keeping conditions described in experiment II. female BDFI mice of the described origin were used to examine the effects of a treatment with the material Bbo-b in case of MXT breast tumor. The place of origin of the tumor cells to be transplanted is the MASON Res. Inst. USA. From the maintained tumor a small piece of 1 m³ volume was implanted subcutane in the interscapular region.

From the animals groups of ten mice were formed as in case of the experiment II, and within each group the individuals were treated equally. The treatment with the material Bbo-b was carried out just as in case of experiment II, i.e. once at the same time each day through 8 days. In case of the positive control the average survival time was 24 days. FIG. 13 shows the survival time in case of the different treated groups. It can be seen that a number of different groups were treated in an identical way, e.g. with 0.15 ml dose groups ip1 and ip2 were equally treated intraperitonially, or the groups po2 and po3 were treated with a dose of 0.3 ml per os. In this tumor type the increase in survival time was about 40%, which was the highest in case of per os treatments.

The tumor mass examinations were carried out on animals in moribund state as described at experiment II, and the results are summarized in FIGS. 14 and 15. The numbers indicated on the horizontal axis relate to the serial numbers of the mice in the particular group, they have no special significance. The vertical sections indicated in the column diagrams relate to the deviations within the associated group. The decrease of the tumor mass was here the most significant also at treatments applied per os.

The examination of 5′-nucleotidase carried out on the experimental animals demonstrated substantial decrease in the treated groups, the analysis of the full experimental data of such examinations has not yet been completed for the time being therefore, they cannot be summarized in the form of a table.

### IV. Experiments with Implanted Acute L₁₂₁₀ Lymphoid Leukemia

The effects of the material Bbo-b was examined in case of acute L₁₂₁₀ lymphoid leukemia. Groups of ten mice were formed from male BDF₁ mice kept as described in connection with Experiments II and III. From the ascites liquid of the DBA/2 mice, in which the acute L₁₂₁₀ lymphoid leukemia has been maintained and from a physiologic saline solution a liquid was made, of which 0.1 ml comprised 10⁶ cells. From that liquid 0.1 ml was injected in an intraperitonial way into each experimental mouse, and in the treated groups the treatments were carried out once a day through 8 days just as in case at Experiments II and III.

FIG. 16 shows the survival data. The average survival time in the positive control group was 9.9 days. It can be seen in FIG. 16 that there is a very substantial difference in the survival time depending on how the material was applied. In response to the intraperitonial and subcutane applications the survival time was 170%, and in contrast thereto the per os application of 0.2 ml dose resulted in a 320% survival time, but in this treated group the differences between the individual animal were large and some of them completely healed. In this tumor type there was no way of determining the relative tumor mass, and the ascites liquid collected in the peritoneal cavity together with the tumors present in the mesenteries constituted the absolute tumor mass. FIG. 17 shows the measured weight of the so determined absolute tumor mass in the moribund groups. The animals that have been alive on the 30^{th} day belonged also to these groups. It can be seen in FIG. 17 that in three of the treated groups no measurable tumor mass was found.

FIG. 18 shows the changing of the activity of 5′-nucleotidase on the 5^{th} and 8^{th} days as well as in moribund state. From the diagrams it can be seen that a substantial improvement took place in all of the treated groups, and in some treated groups normal values were obtained.

In further groups of mice that participated in the experiments I to IV at several different dates histology examinations were carried out. The examinations included the histology analysis of 17 different organs. Based on such histology examinations it can be stated that metastasis has not been formed in any of the examined tumor type. In case of the positive control groups, however, well noticeable metastasis activity was found. These were detected in case of the individual tumor types as follows: in case of the S₁₈₀ sarcoma in the lymphatic glands and in the liver; in case of C₂₆ in the liver and in the mesenteral lymphatic glands; in case of MXT tumor in the liver; and in case of the L₁₂₁₀ in the bone-marrow.

The above experimental results make it likely that the experienced significant improvements will take place also at tumor types not examined here. Examinations covering all types of tumors will be necessary due to their outstanding significance. Nevertheless the above experiments have been sufficiently broad to support the existence of the main effect.

The solution according to the invention can be used efficiently not only for the treatment of tumors but for follow-up care and for preventing the formation of metastases.

Owing to the fact that the two separate fractions which are responsible for the effect but at least the fraction bovin 40 are present in the blood of subjects having tumor, the examination of these fractions can be used for the diagnosis of tumors.

The results obtained with the preparation according to the invention and with its diagnostic potential provide hopes regarding the efficient treatment and diagnosis of tumors. The stock of cattle having leucosis is substantial all over the world, which allows large-scale manufacture. Additionally, there is a possibility for finding a synthetic way for the production of the materials bovin 40 and bovin 300, because the thorough examination of these materials might expectably lead to such manufacture.

1. Pharmaceutical preparation characterized by comprising lipid-free fraction components of the blood plasma of equidae animals with pair number of fingers not endangered lethally by and having leucosis.

2. The method as claimed in claim 17, characterized in that the equidae animals with pair number of fingers being cattle.

3. Pharmaceutical preparation for the treatment of tumors, characterized by comprising a material constituting the difference detectable by electrophoresis between the lipid-free fraction of the blood plasma taken from a cattle having leucosis and the lipid-free fraction of the blood plasma taken from a healthy cattle.

4-14. (canceled)

15. A method according to claim 17 employed for preventing the formation of secondary tumors after a primary tumor.

16. A method according to claim 17 employed for treating any one of S180 sarcoma, C26 colon tumor, MXT breast tumor and acute lymphoid leukemia.

17. A method for the treatment and follow-up care of tumors comprising utilizing a pharmaceutical preparation comprising lipid-free fraction components of the blood plasma of equidae animals with pair number of fingers not endangered lethally by, and having, leucosis.

18. A method for the treatment and follow-up care of tumors comprising utilizing a pharmaceutical preparation comprising a material constituting the difference detectable by electrophoresis between the lipid-free fraction of the blood plasma taken from a cattle having leucosis and the lipid-free fraction of the blood plasma taken from a healthy cattle.
